(19) United States
(12) Patent Application Publication
(10) Pub. No.: US 2025/0295953 A1
(43) Pub. Date: Sep. 25, 2025

(54) ELECTRICAL DEVICE, EXERCISE EVALUATION SYSTEM, METHOD OF EVALUATING EXERCISES, AND RECORDING MEDIUM

(71) Applicant: CASIO COMPUTER CO., LTD., Tokyo (JP)

(72) Inventor: Tomoharu YAMAGUCHI, Tokyo (JP)

(73) Assignee: CASIO COMPUTER CO., LTD., Tokyo (JP)

(21) Appl. No.: 19/072,785

(22) Filed: Mar. 6, 2025

(30) Foreign Application Priority Data
Mar. 22, 2024 (JP) 2024-046643

(51) Int. Cl. A63B 24/00; A63B 71/06; G16H 50/30

(52) U.S. Cl. CPC A63B 24/0062; A63B 71/0622; G16H 50/30; A63B 2220/62; A63B 2220/64; A63B 2220/803

(57) ABSTRACT

An electrical device includes a processor. The processor calculates sums of frequency scores, which are associated with the respective index values regarding exercises of a user and are calculated for individual time points, and fixes the sums of frequency scores as total frequency scores at the individual time points. The processor selects effective time points for evaluation of the exercises of the user, on the basis of the fixed total frequency scores. The processor evaluates the exercises of the user, on the basis of the index values at the selected effective time points.

### CROSS-REFERENCE TO RELATED APPLICATIONS

This application is based upon and claims the benefit of priority under 35 USC 119 of Japanese Patent Application No. 2024-046643 filed on Mar. 22, 2024, the entire disclosure of which, including the description, claims, drawings, and abstract, is incorporated herein by reference in its entirety.

### FIELD OF THE INVENTION

This application relates to an electrical device, an exercise evaluation system, a method of evaluating exercises, and a recording medium.

### BACKGROUND OF THE INVENTION

Unexamined Japanese Patent Application Publication No. 2014-217694 discloses a walking posture meter for evaluating the walking posture of a subject. The walking posture meter includes an acceleration sensor to be mounted on the midline of the waist of the subject, an evaluating unit that repetitively calculates evaluation amounts quantitatively representing the walking posture of the subject on the basis of the outputs from the acceleration sensor at predetermined intervals within a predetermined continuous walking period of 10 minutes or less, and a display operating unit that causes a display screen to display the repetitively calculated evaluation amounts aligned in the chronological order.

### SUMMARY OF THE INVENTION

An electrical device according to a first aspect of the present disclosure includes:
a processor, wherein
the processor executes operations that involve
   calculating sums of frequency scores calculated for individual time points and fixing the sums of frequency scores calculated for the individual time points as total frequency scores at the individual time points, the frequency scores being associated with respective index values regarding exercises of a user,
   selecting, based on the fixed total frequency scores, effective time points for evaluation of the exercises of the user, and
   evaluating, based on the index values at the selected effective time points, the exercises of the user.

An exercise evaluation system according to a second aspect of the present disclosure includes:
a sensor unit to measure physical quantities regarding exercises of a user; and
a processor, wherein
the processor executes operations that involve
   calculating index values regarding the exercises of the user from the physical quantities,
   calculating sums of frequency scores calculated for individual time points and fixing the sums of frequency scores calculated for the individual time points as total frequency scores at the individual time points, the frequency scores being associated with the respective index values regarding the exercises of the user,
   selecting, based on the fixed total frequency scores, effective time points for evaluation of the exercises of the user, and
   evaluating, based on the index values at the selected effective time points, the exercises of the user.

A method of evaluating exercises according to a third aspect of the present disclosure includes:
calculating sums of frequency scores calculated for individual time points and fixing the sums of frequency scores calculated for the individual time points as total frequency scores at the individual time points, the frequency scores being associated with respective index values regarding exercises of a user;
selecting, based on the fixed total frequency scores, effective time points for evaluation of the exercises of the user; and
evaluating, based on the index values at the selected effective time points, the exercises of the user.

A non-transitory computer-readable recording medium according to a fourth aspect of the present disclosure stores a program, the program causing a computer to execute processing comprising:
calculating sums of frequency scores calculated for individual time points and fixing the sums of frequency scores calculated for the individual time points as total frequency scores at the individual time points, the frequency scores being associated with respective index values regarding exercises of a user,
selecting, based on the fixed total frequency scores, effective time points for evaluation of the exercises of the user, and
evaluating, based on the index values at the selected effective time points, the exercises of the user.

### BRIEF DESCRIPTION OF DRAWINGS

A more complete understanding of this application can be obtained when the following detailed description is considered in conjunction with the following drawings, in which:

FIG. 1 is a block diagram illustrating a configuration of an exercise evaluation system according to Embodiment 1;

FIG. 2 illustrates examples of index values calculated by an index value calculator in Embodiment 1;

FIG. 3 is a graph illustrating the index values and the probability densities of occurrence rates of the index values in Embodiment 1;

FIG. 4 illustrates an example of a frequency score table in Embodiment 1;

FIG. 5 illustrates examples of index values and frequency scores in Embodiment 1;

FIG. 6 illustrates examples of index values, frequency scores, and total frequency scores in Embodiment 1;

FIG. 7 is a flowchart illustrating an exercise evaluating process executed by the exercise evaluation system according to Embodiment 1; and

FIG. 8 illustrates a route that a display controller in Embodiment 2 causes a terminal interface to display.

### DETAILED DESCRIPTION OF THE INVENTION

### Embodiment 1

The following describes an exercise evaluation system 100 according to Embodiment 1, with reference to the accompanying drawings. In the drawings, the components identical or corresponding to each other are provided with the same reference symbol. The exercise evaluation system 100 according to Embodiment 1 measures data regarding running exercises of a user, and calculates scores regarding running exercises of the user.

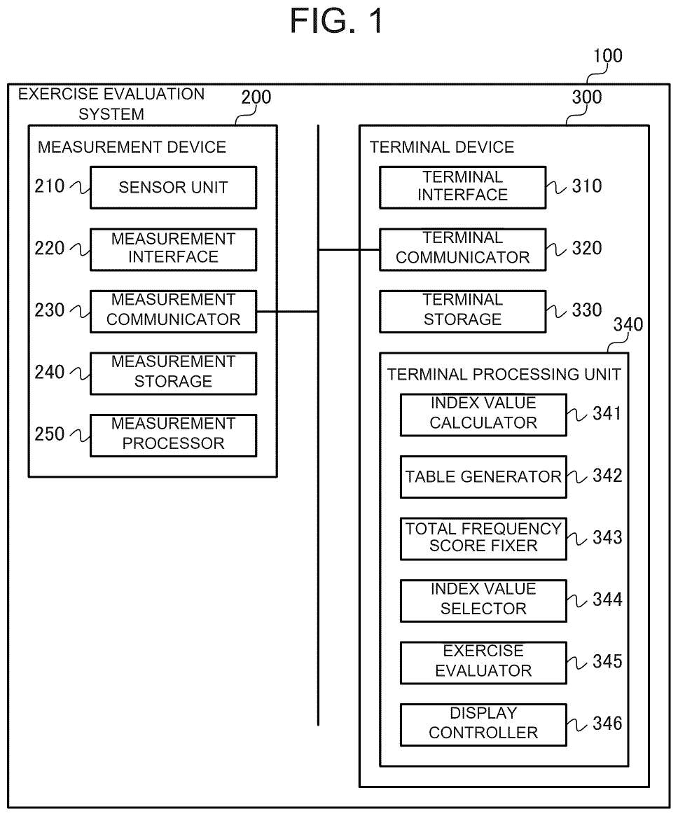

FIG. 1 is a block diagram illustrating a configuration of the exercise evaluation system 100 according to Embodiment 1. As illustrated in FIG. 1, the exercise evaluation system 100 includes a measurement device 200 and a terminal device 300.

The measurement device 200, which is worn by a user who performs running exercises, acquires data regarding the running exercises of the user, and transmits the acquired data to the terminal device 300. The measurement device 200 includes a housing having a clip on one surface. The measurement device 200 is worn by the user when the clip holds the clothing of the user therebetween.

As illustrated in FIG. 1, the measurement device 200 includes a sensor unit 210, a measurement interface 220, a measurement communicator 230, a measurement storage 240, and a measurement processor 250.

The sensor unit 210 measures physical quantities regarding the locations and exercises of the user at individual time points. The sensor unit 210 includes a location sensor that measures the location, an acceleration sensor that measures an acceleration, an angular rate sensor that measures an angular rate, and a magnetic sensor that measures a geomagnetic intensity. The location sensor may include a global positioning system (GPS) receiver that measures the location on the basis of GPS signals, for example. The acceleration sensor may include a semiconductor acceleration sensor or a piezoelectric acceleration sensor, for example. The angular rate sensor may include a vibration gyro sensor, for example. The geomagnetic sensor may include a Hall sensor, a magneto resistance (MR) sensor, or a magneto impedance (MI) sensor, for example. The above-listed sensors are mere examples.

The measurement interface 220 is a user interface including buttons and switches. The measurement interface 220 receives, from the user, an instruction to activate or deactivate the measurement device 200 or an instruction to start or end the data acquisition by the sensor unit 210, and transmits a signal indicating the received instruction to the measurement processor 250.

The measurement communicator 230 is a communication interface that transmits and receives signals to and from the terminal device 300 or other devices. Examples of the measurement communicator 230 include, but are not limited to, a wireless communication interface and a wired communication interface.

The measurement storage 240 is a storage device that stores a program to be executed by the measurement processor 250 and the data measured by the sensor unit 210. Examples of the measurement storage 240 include, but are not limited to, a random access memory (RAM), a flash memory, an erasable programmable ROM (EPROM), and an electrically erasable programmable ROM (EEPROM).

The measurement processor 250 is a circuit that executes the program stored in the measurement storage 240 and thus performs some processes. Examples of the measurement processor 250 include, but are not limited to, a central processing unit (CPU). The measurement processor 250 may also be called the processor 250.

The measurement processor 250 causes the sensor unit 210 to measure physical quantities, causes the measurement storage 240 to store data containing the measured physical quantities and the times of measurement in the form of exercise data, and transmits the exercise data to the terminal device 300 via the measurement communicator 230.

The terminal device 300 calculates scores regarding running exercises of the user on the basis of the data regarding the running exercises of the user acquired from the measurement device 200, and presents the calculated scores to the user. Examples of the terminal device 300 include, but are not limited to, electrical device such as a smartphone.

As illustrated in FIG. 1, the terminal device 300 includes a terminal interface 310, a terminal communicator 320, a terminal storage 330, and a terminal processing unit 340.

The terminal interface 310 is a user interface including a touch panel that displays visual information and receives a manipulation of the user, a speaker or earphone that presents acoustic information, buttons, and switches. The terminal interface 310 transmits a signal indicating the received manipulation of the user to the terminal processing unit 340.

The terminal communicator 320 is a communication interface that transmits and receives signals to and from the measurement device 200 or other devices. Examples of the terminal communicator 320 include, but are not limited to, a wireless communication interface and a wired communication interface.

The terminal storage 330 stores a program to be executed by the terminal processing unit 340, data to be used in the program, exercise data, and scores. Examples of the terminal storage 330 include, but are not limited to, a RAM, a flash memory, an EPROM, and an EEPROM.

The terminal processing unit 340 is a circuit that executes the program stored in the terminal storage 330 and thus performs some processes. The terminal processing unit 340 includes an index value calculator 341, a table generator 342, a total frequency score fixer 343, an index value selector 344, an exercise evaluator 345, and a display controller 346. Examples of the terminal processing unit 340 include, but are not limited to, a CPU. The terminal processing unit 340 is also called the terminal processor 340.

The terminal processing unit 340 acquires the exercise data measured by the sensor unit 210 of the measurement device 200 via the terminal communicator 320, and causes the exercise data to be stored into the terminal storage 330. The index value calculator 341 calculates index values regarding running exercises of the user at predetermined intervals, from the physical quantities contained in the exercise data stored in the terminal storage 330, and causes the calculated index values to be stored into the terminal storage 330. Examples of the index values calculated by the index value calculator 341 include, but are not limited to, vertical motions, turning motions of the pelvis, strides, and grounded periods of a foot.

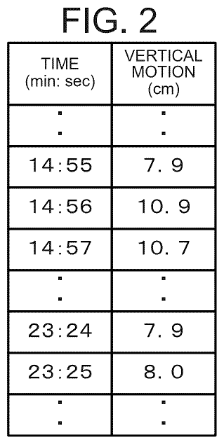

FIG. 2 illustrates examples of the index values calculated by the index value calculator 341. As illustrated in FIG. 2, the index value calculator 341 calculates index values regarding running exercises of the user at predetermined intervals, and causes the calculated index values to be stored into the terminal storage 330. The index value calculator 341 in the example illustrated in FIG. 2 calculates an index value of vertical motion every one second.

The table generator 342 acquires the index values calculated by the index value calculator 341 from the terminal storage 330, defines ranges of index value, generates a frequency score table containing the defined ranges of index value and frequency scores of the index values encompassed in the ranges in association with each other, and causes the generated frequency score table to be stored into the terminal storage 330. The table generator 342 may define the ranges of index value on the basis of the average and the standard deviation of the index values, assuming that the index values occur in accordance with the normal distribution. The table generator 342 assigns a lower frequency score to a range closer to the average, and assigns a higher frequency score to a range farther from the average.

The table generator 342 defines the ranges of index value on the basis of the occurrence rates of the index values within the ranges of index value. Specifically, the table generator 342 defines the ranges of index value so as to approximately equalize the occurrence rates of the index values encompassed in the individual ranges.

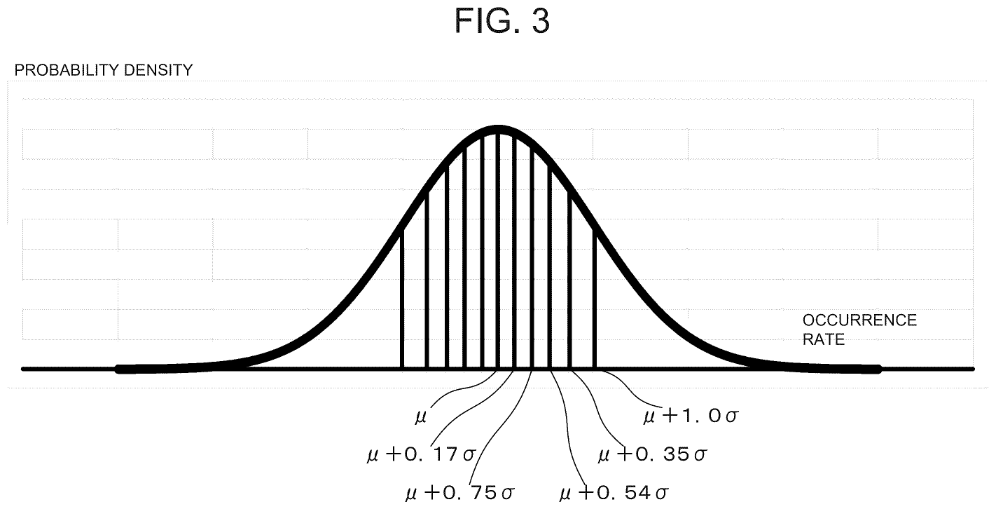

FIG. 3 is a graph illustrating the index values and the probability densities of occurrence rates of the index values. In FIG. 3, the horizontal axis represents an index value x in terms of an average u and a standard deviation a, and the vertical axis represents a probability density of occurrence rate of an index value. The index values in the example illustrated in FIG. 3 are assumed to occur in accordance with the normal distribution.

In FIG. 3, the area represented by the inequality |x−μ|≤0.17σ is approximately equal to the area represented by the inequality 0.17σ<|x−μ|≤0.35σ. In other words, the occurrence rate of an index value x satisfying the inequality |x−μ|≤0.17σ is approximately equal to the occurrence rate of an index value x satisfying the inequality 0.17σ<|x−μ|≤0.35σ. The same holds true for the other ranges. The table generator 342 thus defines the ranges of index value so as to approximately equalize the areas of the individual ranges of the graph, in other words, to approximately equalize the occurrence rates of the index values within the individual ranges.

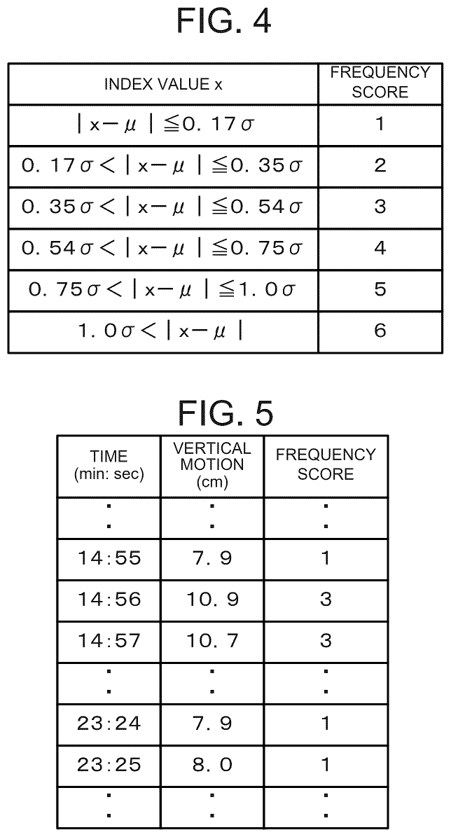

FIG. 4 illustrates an example of a frequency score table. As illustrated in FIG. 4, the frequency score table contains the ranges of index value, and frequency scores associated with the respective ranges. The ranges of index value x can be expressed in terms of the average and the standard deviation a. For example, the ranges of index value x are expressed by the inequations |x−μ|≤0.17σ, 0.17σ<|x−μ|≤0.35σ, 0.35σ<|x−μ|≤0.54σ, 0.54σ<|x−μ|≤0.75σ, 0.75σ<|x−μ|≤1.0σ, and 1.0σ<|x−μ|, which are associated with the frequency scores 1, 2, 3, 4, 5, and 6, respectively. These ranges of index value and frequency scores are mere examples.

The total frequency score fixer 343 acquires, from the terminal storage 330, the index value regarding running exercises of the user calculated by the index value calculator 341 and the frequency score table, and determines which range of index value in the frequency score table encompasses the acquired index value. The total frequency score fixer 343 then fixes, as the frequency score of the acquired index value, the frequency score associated in the frequency score table with the range of index value determined to encompass the index value, and causes the fixed frequency score to be stored into the terminal storage 330. The total frequency score fixer 343 fixes the frequency scores of all the index values calculated by the index value calculator 341 and stored in the terminal storage 330, on the basis of the frequency score table.

FIG. 5 illustrates examples of index values and frequency scores. The example illustrated in FIG. 5 involves the fixation of the frequency scores of the index values of vertical motion. As illustrated in FIG. 5, the total frequency score fixer 343 fixes the frequency scores of the index values of vertical motion, each calculated by the index value calculator 341 every one second, on the basis of the frequency score table.

The total frequency score fixer 343 acquires the frequency scores of all the index values from the terminal storage 330, calculates the sums of the acquired frequency scores of all the index values for the individual time points, fixes the sums as the total frequency scores at the individual time points, and then causes the fixed total frequency scores to be stored into the terminal storage 330.

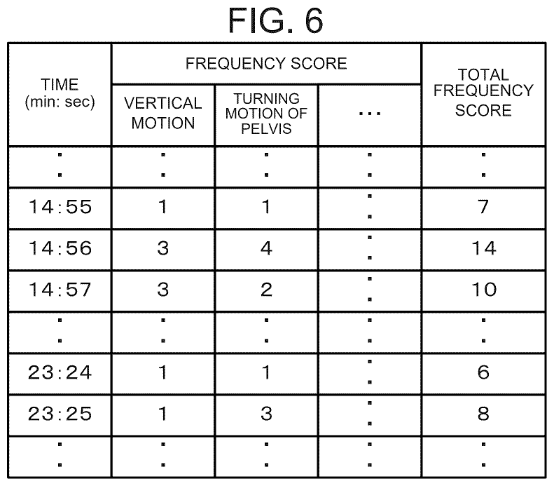

FIG. 6 illustrates examples of index values, frequency scores, and total frequency scores. The example illustrated in FIG. 6 involves the calculation of index values and the fixation of frequency scores, with respect to parameters, such as vertical motions and turning motions of the pelvis. As illustrated in FIG. 6, the total frequency score fixer 343 calculates the sum of the frequency scores of the index values for each time point, and fixes the total frequency score at the time point.

The index value selector 344 compares the total frequency scores at the individual time points fixed by the total frequency score fixer 343 with a threshold score, and selects, as effective time points, the time points each providing a total frequency score equal to or lower than the threshold score. The index value selector 344 then causes the selected effective time points to be stored into the terminal storage 330. The effective time points are used to evaluate the exercises of the user, as described below. The index value selector 344 may apply the minimum frequency score as the threshold score. In other words, the index value selector 344 may select the time point of which all the frequency scores are 1 to be fixed as the total frequency score.

The exercise evaluator 345 calculates exercise scores for evaluation of the exercises of the user, with respect to the index values at the effective time points selected by the index value selector 344 among the index values calculated by the index value calculator 341 and stored in the terminal storage 330. The exercise evaluator 345 then causes the calculated exercise scores to be stored into the terminal storage 330. For example, the calculation provides a high exercise score when the index values of vertical motion calculated by the index value calculator 341 indicate appropriate running exercises of the user. In contrast, the calculation provides a low exercise score when the index values of excessively large index values of vertical motion indicate inappropriate exercises of the user.

The display controller 346 acquires the exercise scores stored in the terminal storage 330, and causes the terminal interface 310 to display the acquired exercise scores.

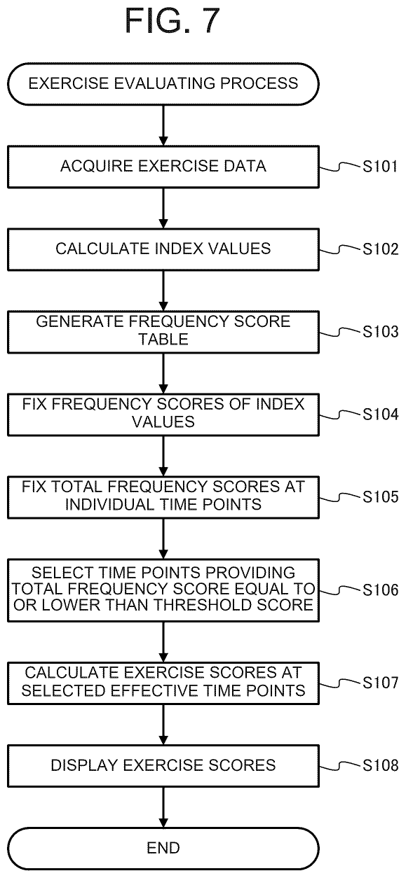

FIG. 7 is a flowchart illustrating an exercise evaluating process executed by the exercise evaluation system 100 according to Embodiment 1. The exercise evaluating process is described below with reference to the flowchart of FIG. 7. The measurement device 200 is assumed to be worn by the user and measuring exercise data in advance of the exercise evaluating process.

At the start of the exercise evaluating process, the terminal device 300 acquires the exercise data measured by the sensor unit 210 of the measurement device 200, via the terminal communicator 320 (Step S101).

After the acquisition of the exercise data, the index value calculator 341 calculates index values regarding running exercises of the user at predetermined intervals, from the physical quantities contained in the acquired exercise data (Step S102).

After the calculation of the index values by the index value calculator 341, the table generator 342 defines the ranges of index value, and generates a frequency score table containing the defined ranges of index value and frequency scores of the index values encompassed in the ranges, in association with each other (Step S103).

After the generation of the frequency score table by the table generator 342, the total frequency score fixer 343 determines which range of index value in the frequency score table encompasses the index value calculated by the index value calculator 341, and fixes, as the frequency score of the acquired index value, the frequency score associated in the frequency score table with the range of index value determined to encompass the index value (Step S104).

After the fixation of the frequency scores of all the index values on the basis of the frequency score table, the total frequency score fixer 343 calculates the sums of the fixed frequency scores of all the index values for the individual time points, and fixes total frequency scores at the individual time points (Step S105).

After the fixation of the total frequency scores by the total frequency score fixer 343, the index value selector 344 compares the total frequency scores at the individual time points with the threshold score, and selects, as effective time points, the time points each providing a total frequency score equal to or lower than the threshold score (Step S106).

After the selection of the effective time points each providing a total frequency score equal to or lower than the threshold score by the index value selector 344, the exercise evaluator 345 calculates exercise scores for evaluation of the exercises of the user with respect to the index values at the selected effective time points (Step S107).

After the calculation of the exercise scores by the exercise evaluator 345, the display controller 346 causes the terminal interface 310 to display the exercise scores calculated by the exercise evaluator 345 (Step S108), followed by termination of the exercise evaluating process.

The exercise evaluation system 100 according to Embodiment 1, which has the above-described configuration and executes the exercise evaluating process, can evaluate the exercises of the user without being affected by motions deviated from the average exercises of the user.

For example, the user during running sometimes makes sudden motions, such as a slow zig-zag running motion through a crowd, and a jumping motion over a puddle. If the data on such motions is contained in the data for evaluation of the exercises of the user, an existing system may fail to appropriately evaluate the whole exercises of the user. In contrast, the exercise evaluation system 100 according to Embodiment 1 fixes the total frequency scores, and calculates exercise scores for evaluation of the exercises of the user with respect to the index values at the time points each providing a total frequency score equal to or lower than the threshold score. The exercise evaluation system 100 can thus evaluate the average exercises of the user except for sudden motions of the user.

The index values regarding exercises of the user are expected to occur in accordance with the normal distribution. The exercise evaluation system 100 according to Embodiment 1 defines the ranges of index value so as to approximately equalize the occurrence rates of the index values in the individual ranges, on the basis of the average and the standard deviation of the index values. The exercise evaluation system 100 can thus appropriately select the data on the average exercises of the user and evaluate the exercises of the user.

Exercises of a user can vary with time or place. Thus, a portion of a series of exercises of the user may be selected, and the exercises of the user may be evaluated based on the selected portion. According to the walking posture meter disclosed in Unexamined Japanese Patent Application Publication No. 2014-217694, the evaluation amounts quantitatively representing the walking posture of the user can be calculated. However, the walking posture meter is not designed to, in a case where multiple pieces of data on the exercises of the user are used, collect the multiple pieces of data together, and effectively select portions that represent the exercises of the user.

The present disclosure is made with the view of the above situation, and an objective of the present disclosure is to provide an electrical device, an exercise evaluation system, a method of evaluating exercises, and a program capable of selecting a portion that represents the exercises of the user from the series of exercises of the user to evaluate.

### Embodiment 2

The following describes an exercise evaluation system 100 according to Embodiment 2, with reference to the accompanying drawings. The exercise evaluation system 100 according to Embodiment 2 has the configuration identical to that of the exercise evaluation system 100 according to Embodiment 1 illustrated in FIG. 1. The exercise evaluation system 100 according to Embodiment 2 displays a route of exercises of the user on the basis of the total frequency scores.

The display controller 346 in Embodiment 2 acquires the exercise data and the total frequency scores from the terminal storage 330, and causes the terminal interface 310 to display a running route of the user in various manners depending on total frequency scores, on the basis of the times and/or locations contained in the exercise data and the total frequency scores. The display controller 346 is able to display the running route in various colors, thicknesses, patterns, or types depending on total frequency scores. The display controller 346 may depict the route on a map.

The terminal interface 310 may receive a manipulation of the user for selecting at least a segment of the displayed running route of the user. The manipulation for selecting a segment of the route is achieved in response to a touching manipulation of the user on a portion of the screen representing the route displayed on the touch panel. This configuration is, however, a mere example.

The index value selector 344 may acquire the signal indicating the route segment selected by the user from the terminal interface 310, and select the time points associated with the selected route segment. In other words, the index value selector 344 may select time points on the basis of the total frequency scores, by selecting the time points associated with the route segment in response to selection of a segment of the route displayed on the basis of the total frequency scores.

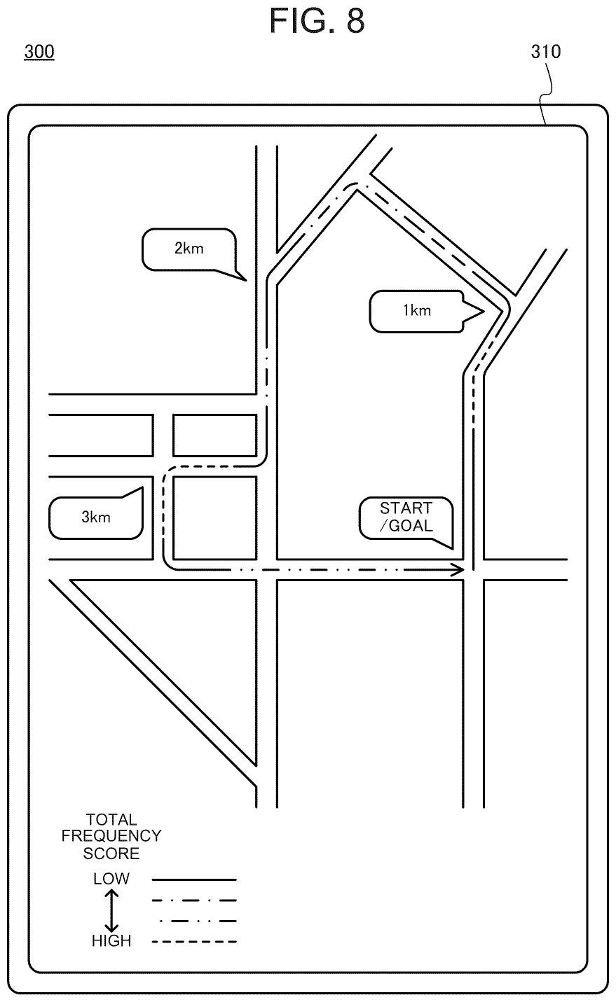

FIG. 8 illustrates the route that the display controller 346 causes the terminal interface 310 to display. As illustrated in FIG. 8, the display controller 346 displays a map of the region containing the running route on the touch panel of the terminal interface 310, and superimposes the running route of the user on the map in various manners depending on total frequency scores. In the example illustrated in FIG. 8, the route is displayed in various types of lines. The display controller 346 may also display explanatory notes containing the start or goal, distances, time points, and total frequency scores.

The exercise evaluation system 100 according to Embodiment 2, which has the above-described configuration and executes the exercise evaluating process, brings about the same effects as the exercise evaluation system 100 according to Embodiment 1.

The exercise evaluation system 100 according to Embodiment 2 displays the route of the user in various manners depending on total frequency scores, and can thus allow the user to readily recognize the segments of the average exercises or the segments of sudden motions. The exercise evaluation system 100 according to Embodiment 2 enables the user to select a route segment on the basis of the total frequency scores. By allowing the user to select the route segment, the exercise evaluation system 100 can evaluate the exercises in only the route segment required to be evaluated by the user.

### Modifications

The above-described embodiments of the present disclosure are mere examples and not to be construed as limiting the application scope of the present disclosure. That is, the embodiments of the present disclosure may be provided with various modifications, and any modified embodiment can be encompassed in the scope of the present disclosure.

Although the exercise evaluation system 100 measures data regarding running exercises of the user and calculates scores regarding running exercises of the user, this configuration is a mere example. The exercise evaluation system 100 may calculate scores regarding physical exercises including dances and sports, such as swimming, ball games, and walking, other than running.

The sensor unit 210 includes the location sensor that measures the location, the acceleration sensor that measures an acceleration, the angular rate sensor that measures an angular rate, and the magnetic sensor that measures a geomagnetic intensity. This configuration is, however, a mere example. The sensor unit 210 may exclude any of the above-listed sensors, or may include any sensor that measures physical quantities regarding the locations and exercises of the user other than the above-listed sensors.

Although the table generator 342 defines the ranges of index value so as to approximately equalize the occurrence rates of the index values encompassed in the individual range, this configuration is a mere example. The table generator 342 may define the ranges of index value in any procedure, for example, by making the index values encompassed in a certain range to have a high or low occurrence rate.

Although the table generator 342 assigns a lower frequency score to a range closer to the average and assigns a higher frequency score to a range farther from the average, this configuration is a mere example. The table generator 342 may use any combination of ranges and frequency scores, for example, by assigning a higher frequency score to a range closer to the average and assigning a lower frequency score to a range farther from the average.

Although the terminal processing unit 340 includes the table generator 342 that generates a frequency score table, this configuration is a mere example. The terminal processing unit 340 may include a frequency score fixer that fixes frequency scores using the ranges of index value, without generating a frequency score table. The frequency score fixer may define the ranges of index value and fix the frequency scores, like the table generator 342, except for the generation of a frequency score table. The table generator 342 may include the frequency score fixer. Alternatively, the frequency score fixer may include the table generator 342.

This frequency score fixer or the index value calculator 341 may be excluded from the terminal device 300. In other words, the fixation of frequency scores or the calculation of index values may be performed by a unit outside the terminal device 300.

Although the total frequency score fixer 343 fixes the total frequency scores by calculating the sums of the frequency scores of all the index values for the individual time points, this configuration is a mere example. The total frequency score fixer 343 may fix the total frequency scores on the basis of the frequency scores of some of the index values.

Although the index value selector 344 compares the total frequency scores fixed by the total frequency score fixer 343 with the threshold score, and selects, as effective time points, the time points each providing a total frequency score equal to or lower than the threshold score, this configuration is a mere example. The index value selector 344 may select the time points each providing a total frequency score equal to or higher than a threshold score, and use any selection procedure depending on results of the comparison.

The index value selector 344 may vary the threshold score to be compared with the total frequency scores. In some cases, the exercises of the user may have a larger dispersion due to a high fatigue level of the user, or the unevenness of terrain, for example. In other cases, the exercises of the user may have a smaller dispersion due to constant exercise conditions. The index value selector 344 in such cases selects the time points while varying the threshold score, and can thus more appropriately evaluate the exercises of the user.

Although the exercise evaluator 345 calculates exercise scores for evaluation of the exercises of the user, this configuration is a mere example. The exercise evaluator 345 may evaluate the exercises of the user by any procedure other than the calculation of scores.

Although the measurement device 200 is worn by the user and measuring exercise data in advance of the exercise evaluating process, this configuration is a mere example. The terminal device 300 may acquire exercise data other than the data measured by the measurement device 200.

Although the terminal interface 310 in Embodiment 2 receives a manipulation of the user for selecting a segment of the displayed running route of the user, this configuration is a mere example. The terminal interface 310 may receive a manipulation of the user for selecting any element, such as time period, place, and type of exercise, other than the route segment.

The exercise evaluation system 100 may also be achieved by a single device having both of the functions of the measurement device 200 and the functions of the terminal device 300. In other words, the measurement device 200 and the terminal device 300 may be the identical device.

As well as a measurement device or electrical device preliminarily having a configuration for performing the functions according to the present disclosure, a program may be applied to cause an existing measurement device or electrical device to function respectively as the measurement device or electrical device according to the present disclosure. That is, a program for achieving the functions of the measurement device or electrical device illustrated in the embodiments and the modifications may be applied so as to be executable by the CPU for controlling the existing measurement device or electrical device, for example, and thereby cause the existing measurement device or electrical device to function respectively as the measurement device or electrical device according to the present disclosure.

Such a program can be applied in any procedure. For example, the program may be stored in a non-transitory computer-readable recording medium, such as flexible disk, compact disc ROM (CD-ROM), digital versatile disc ROM (DVD-ROM), or memory card and then applied. Alternatively, the program may be superimposed on a carrier wave and applied via a communication medium, such as the Internet. For example, the program may be posted on a bulletin board system (BBS) on a communication network and then distributed. The program may be activated and executed under the control of an operating system (OS), like other application programs, thereby achieving the above-described process.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

1. An electrical device, comprising:
a processor, wherein
the processor executes operations that involve
calculating sums of frequency scores calculated for individual time points and fixing the sums of frequency scores calculated for the individual time points as total frequency scores at the individual time points, the frequency scores being associated with respective index values regarding exercises of a user,
selecting, based on the fixed total frequency scores, effective time points for evaluation of the exercises of the user, and
evaluating, based on the index values at the selected effective time points, the exercises of the user.

2. The electrical device according to claim 1, wherein
the processor further executes operations that involve
calculating the index values regarding the exercises of the user, and
fixing the frequency scores using ranges of the index values, and
the fixing of the sums of frequency scores calculated for the individual time points as the total frequency scores involves calculating sums of frequency scores calculated for the individual time points, the frequency scores being fixed using the ranges of the index values and being associated with the respective index values.

3. The electrical device according to claim 1, wherein the frequency scores are fixed by defining ranges of the index values in terms of an average and a standard deviation of the index values.

4. The electrical device according to claim 2, wherein the frequency scores are fixed by defining the ranges of the index values in terms of an average and a standard deviation of the index values.

5. The electrical device according to claim 1, wherein the frequency scores are fixed by defining ranges of the index values in accordance with occurrence rates of the index values encompassed in the ranges of the index values.

6. The electrical device according to claim 2, wherein the frequency scores are fixed by defining the ranges of the index values in accordance with occurrence rates of the index values encompassed in the ranges of the index values.

7. The electrical device according to claim 1, wherein the selecting of the effective time points involves selecting the effective time points based on a result of comparison of the total frequency scores with a threshold score, the total frequency scores being fixed in the fixing of the sums as the total frequency scores.

8. The electrical device according to claim 2, wherein the selecting of the effective time points involves selecting the effective time points based on a result of comparison of the total frequency scores with a threshold score, the total frequency scores being fixed in the fixing of the sums as the total frequency scores.

9. The electrical device according to claim 1, further comprising:
a display controller to display, based on time points or locations regarding the exercises of the user, a time period or a route that the user has passed through, wherein
the time period or the route is displayed in various manners depending on the total frequency scores.

10. The electrical device according to claim 2, further comprising:
a display controller to display, based on time points or locations regarding the exercises of the user, a time period or a route that the user has passed through, wherein
the time period or the route is displayed in various manners depending on the total frequency scores.

11. The electrical device according to claim 9, wherein the selecting of the effective time points involves selecting, as the effective time points, time points selected in response to a manipulation of the user for selecting the time period or the route displayed by the display controller.

12. An exercise evaluation system, comprising:
a sensor unit to measure physical quantities regarding exercises of a user; and
a processor, wherein
the processor executes operations that involve
calculating index values regarding the exercises of the user from the physical quantities,
calculating sums of frequency scores calculated for individual time points and fixing the sums of frequency scores calculated for the individual time points as total frequency scores at the individual time points, the frequency scores being associated with the respective index values regarding the exercises of the user,
selecting, based on the fixed total frequency scores, effective time points for evaluation of the exercises of the user, and
evaluating, based on the index values at the selected effective time points, the exercises of the user.

13. A method of evaluating exercises, the method comprising:
calculating sums of frequency scores calculated for individual time points and fixing the sums of frequency scores calculated for the individual time points as total frequency scores at the individual time points, the frequency scores being associated with respective index values regarding exercises of a user;
selecting, based on the fixed total frequency scores, effective time points for evaluation of the exercises of the user; and
evaluating, based on the index values at the selected effective time points, the exercises of the user.

14. The method of evaluating exercises according to claim 13, further comprising:
calculating the index values regarding the exercises of the user; and
fixing the frequency scores using ranges of the index values, wherein
the fixing of the sums of frequency scores calculated for the individual time points as the total frequency scores involves calculating sums of frequency scores calculated for the individual time points, the frequency scores being fixed using the ranges of the index values and being associated with the respective index values.

15. The method of evaluating exercises according to claim 13, wherein the frequency scores are fixed by defining ranges of the index values in terms of an average and a standard deviation of the index values.

16. The method of evaluating exercises according to claim 14, wherein the frequency scores are fixed by defining the ranges of the index values in terms of an average and a standard deviation of the index values.

17. The method of evaluating exercises according to claim 13, wherein the frequency scores are fixed by defining ranges of the index values in accordance with occurrence rates of the index values encompassed in the ranges of the index values.

18. The method of evaluating exercises according to claim 14, wherein the frequency scores are fixed by defining the ranges of the index values in accordance with occurrence rates of the index values encompassed in the ranges of the index values.

19. A non-transitory computer-readable recording medium storing a program, the program causing a computer to execute processing comprising:
calculating sums of frequency scores calculated for individual time points and fixing the sums of frequency scores calculated for the individual time points as total frequency scores at the individual time points, the frequency scores being associated with respective index values regarding exercises of a user;
selecting, based on the fixed total frequency scores, effective time points for evaluation of the exercises of the user; and
evaluating, based on the index values at the selected effective time points, the exercises of the user.
